(19) United States
(12) Patent Application Publication
(10) Pub. No.: US 2008/0119944 A1
(43) Pub. Date: May 22, 2008

(54) Means for Fixing Prostheses, Method of Inserting a Prosthesis Into a Grain Bed and a Prosthesis Insertion Unit

(76) Inventors: Lars Bruce, Viken (SE); Bengt Mjoberg, Ystad (SE); Lars Magnus Bjursten, Limhamn (SE)

Correspondence Address: SMITH, GAMBRELL & RUSSELL, 1130 CONNECTICUT AVENUE, N.W., SUITE 1130, WASHINGTON, DC, 20036, US

(21) Appl. No.: 11/663,266

(22) PCT Filed: Sep. 21, 2005
(86) PCT No.: PCT/SE05/01384

§ 371 Date: Nov. 9, 2007

Related U.S. Application Data
(60) Provisional application No. 60/611,389, filed on Sep. 21, 2004.

(30) Foreign Application Priority Data
Sep. 21, 2004 (SE) 0402277-8

(51) Int. Cl. A61F 2/30; A61B 19/00

(52) U.S. Cl. 623/23.44; 128/898

(57) ABSTRACT

A method and an insertion unit for inserting an elongate prosthesis into a prosthesis fixing device. This device comprises a bed of grains and biocompatible material, and a vibrating tool is used for insertion, the vibration head of which performs an oscillating motion back and forth about a swivelling axis (3′). According to the invention, said axis (3r) of the vibrating tool essentially coincides with the longitudinal axis (I1) of the elongate prosthesis (1). The method and the insertion unit provide a prosthesis fixing device consisting of said bed, in which the grains are evenly packed along the length of the prosthesis.

The invention relates on the one hand to a method for inserting an elongate prosthesis into a grain bed and, on the other, a prosthesis insertion unit according to the preamble of claims 1 and 2, respectively.

The introduction of bone cement in the 1960s for fixing joint prostheses resulted in a dramatic improvement of the surgery results. Bone cement fills irregularities and takes care of insufficient fit between bone and prosthesis that arises due to varying anatomy or surgical inaccuracy. Bone cement thus provides an increased contact surface and a better and more evenly distributed load. However, bone cement also suffers from drawbacks. Thus, it comprises a toxic and allergenic monomer component, monomer methyl methacrylate, which leaks out from the cement composition and can enter the blood vessel during surgery, thus causing a fall in blood pressure in the patient, which requires cortisone treatment during the actual cementing. Bone cement hardens in the course of 10-15 min. sometimes at a relatively high temperature (due to, inter alia, the thickness of the cement), above 70° C. is not unusual, which may result in thermal damage to the bone. Moreover bone cement is brittle and there is a risk of fatigue fractures over time.

In the 1970s it was observed that cemented prostheses which initially seemed to be well fixed could sometimes come loose after several years. It was considered—and many still do consider—that such coming loose might be caused by cement particles. A large number of uncemented prostheses were marketed and expected to establish direct contact between bone tissue and prosthesis (osseointegration). Uncemented prosthesis fixation can be achieved by an accurate surgical technique and wedging of the prosthesis into the bone. However, this method has been found not perfectly reliable, and therefore the prosthesis has been provided with a porous surface or coated with hydroxyapatite in order to improve the possibility of bone ingrowth. The clinical results have varied from quite poor to good.

A serious problem in reoperation of loosened hip prostheses is the replacement of the bone that has been lost in connection with the loosening of the prosthesis. The common technique involves the use of pulverised bone. If possible, the patient's own bone is used, but in consideration of the fact that in most cases large amounts of material are required, frozen bone collected from other patients must be used. This involves a risk of transfer of infectious matter, such as HIV and hepatite virus. Various artificial bone substitutes have been tested over the years with different degrees of success. In reoperation, the prosthesis is fixed with cement in the bone-packed cavity. The clinical results have been good so far, but the intended bone ingrowth of the prostheses did not occur.

U.S. Pat. No. 5,015,256 discloses a different technique for fixing a prosthesis in a bone cavity. This technique does not use cement or packed pulverised bone but is instead based on the use of porous, irregular plastic grains consisting of biologically compatible material, having a size of preferably 0.5-2 mm, preferably of titanium, between the prosthesis (both for new operation and reoperation) and the inner wall of the bone cavity. The porous titanium grains induce bone ingrowth from the inner wall, through and between the grains and to the prosthesis and thus help to anchor the prosthesis in the bone cavity. A further advantage of the use of such grains is the elimination of the risk of infection. Moreover, they are not resorbed like packed pulverised bone. As a result, the conditions of maintaining the stability between bone and prosthesis, which is critical for ingrowth, will be significantly improved compared with natural bone as filling material.

According to U.S. Pat. No. 5,015,256, the reamed canal in the shaft of a femur is filled with titanium grains, and a femoral prosthesis is driven into the bed of titanium grains by a vibrating tool, a pneumatically driven, oscillating bone saw which strikes on the prosthesis head. In one embodiment, the initial vibration of the prosthesis stem occurs in an approximately horizontal circular arc back and forth at a first vibration frequency, thereby making the grains in the bed of grains fluidise, after which a motion occurs back and forth in the longitudinal direction of the stem at a second lower frequency, thereby packing the grains and locking them to each other. After vibration, a striking tool (hammer) is used to drive the prosthesis to its final locked position in the femur.

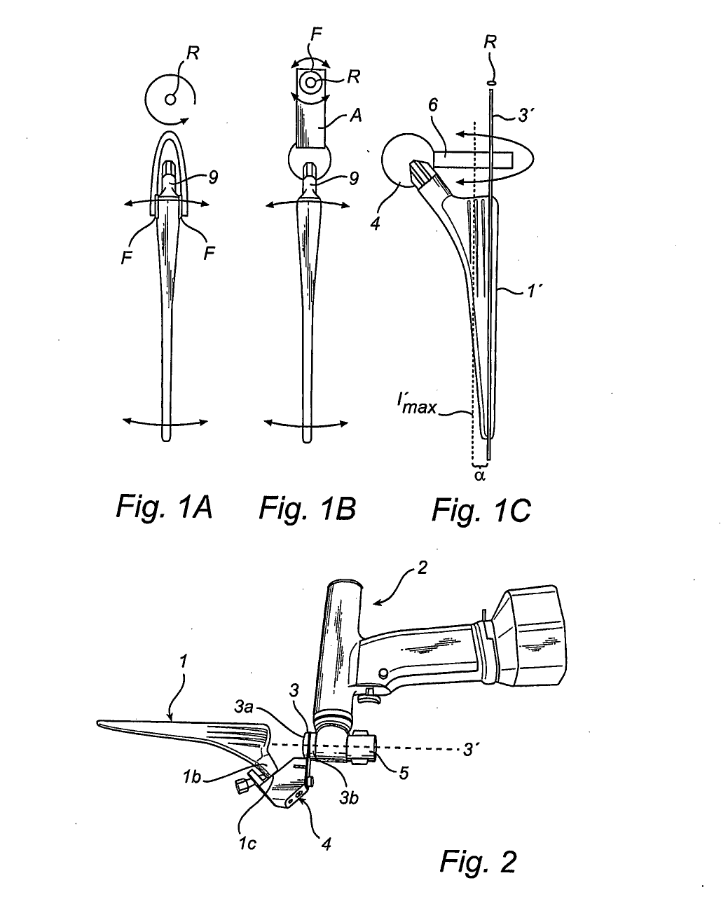

Although the just described process of inserting a femoral prosthesis into the femur in many cases functions in a satisfactory manner, surgeons applying the technique sometimes experience that it is imprecise, which is demonstrated by the prosthesis being incorrectly positioned in a first attempt to drive down the prosthesis, so that the prosthesis must be removed and repositioned to be driven down once more by vibration and a final stroke. Moreover, great force is required to drive down the prosthesis to its final position in the femur. A further fact that can be experienced as slightly inconvenient in this prior-art technique is that the fluidised grains tend to be scattered about at the site of surgery, thus necessitating cleaning of the wound. Further problems with this technique will be described below with reference to FIGS. 1A and 1B.

The object of the invention is to remedy, based on a technique involving driving a prosthesis down into a body cavity essentially filled with grains, the just described problems with imprecise positioning, mobilisation of forces for the final stroke and cleaning of the site of surgery owing to grains being scattered about.

In the development work behind the invention, it was discovered that the problems of prior-art technique according to U.S. Pat. No. 5,016,256 were caused on the one hand by the relationship between the vibration axis (swivelling axis) of the vibrating tool and the longitudinal axis of the prosthesis and, on the other hand, impact force being used continuously to drive the prosthesis down into the grain bed. To illustrate this, reference is made to FIGS. 1A and 1B, where FIG. 1A shows the above-discussed technique according to the US patent. A fork attached to a rod-shaped vibrating tool loosely holds the proximal portion of the stem and the neck of a femoral prosthesis to apply a stroke to the prosthesis, which means that during vibration about the axis R, the prosthesis stem oscillates about an axis back and forth perpendicular to the longitudinal direction of the prosthesis stem. FIG. 1B illustrates a vibrating tool with an adapter A which firmly holds the conical head of a femoral prosthesis so that the vibration axis of the vibrating tool extends at right angles to the longitudinal axis of the prosthesis. Also in this case, the prosthesis oscillates back and forth about the vibration axis R perpendicular to the longitudinal direction of the prosthesis stem. (This technique of vibration does not belong to prior art). In both cases, for instance, the prosthesis turns counterclockwise as the vibrator turns clockwise. In the initial stage of driving down the prosthesis into the grain bed, this causes a tendency of the grains being scattered about, and also difficulties in attaching the prosthesis to the vibrating tool. High friction and, resulting therefrom, heat generation between fork and prosthesis are marked at the points F. In the vibrating technique with the stable anchoring of the vibrating tool to the cone of the prosthesis according to FIG. 1B, the connection between the adapter A and vibrating tool is instead subjected to these opposite motions, here also resulting initially in the grains tending to be scattered about, and also considerable friction (ring F marked in grey around the rotation axis R), with the ensuing generation of heat. When the tip of the prosthesis has been moved down a distance into the grain bed and, thus, the motions of the tip have been dampened, the upper part of the prosthesis will start to swivel, which also causes a risk of the grains being scattered about.

The lessons of the development work described above have led to the invention, by which the inventive objects are achieved, implying that the prosthesis is to be rigidly connected to a vibrating tool and the prosthesis is to be driven down into the grain bed with an oscillating spiral motion, the vibration axis of the vibrating tool essentially coinciding with the longitudinal axis of the prosthesis.

In addition, a substantially uniform packing of the grains along the length of the prosthesis stem is also achieved, which increases the extraction resistance of the prosthesis, relative to the prior-art techniques according to FIG. 1A and FIG. 1B, which results in the fact that, while the lower part of the grain bed is compact (requiring great forces for the prosthesis stem to be driven down), the upper part of the grain bed is fluffy. By substantially uniform is meant substantially no density gradient over the prosthesis stem in the bed.

The principle of the inventive technique is illustrated in FIG. 1C.

More specifically, the objects above are achieved by a method and a prosthesis insertion unit as claimed in claim 1, advantageous embodiments having the features stated in the dependent claims.

The invention will now be described in more detail with reference to the accompanying drawings, in which

FIG. 1A illustrates a prosthesis insertion technique according to prior art (U.S. Pat. No. 5,015,256),

FIG. 1B illustrates a previously not known prosthesis insertion technique employed in the work with the development of the invention,

FIG. 1C illustrates a prosthesis insertion technique according to the invention,

FIG. 2 illustrates a configuration in perspective of the prosthesis insertion unit according to the invention,

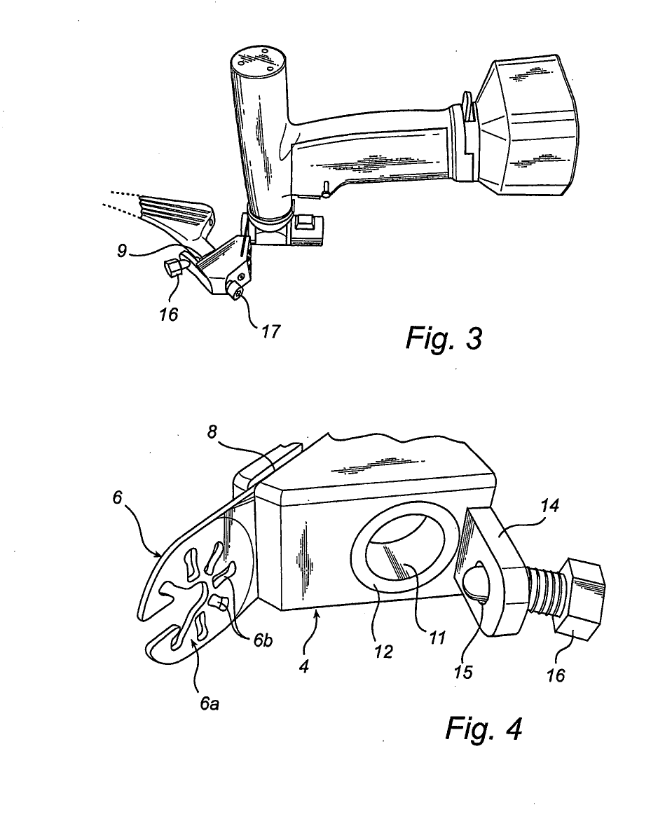

FIG. 3 shows another configuration in perspective of the prosthesis insertion unit according to the invention,

FIG. 4 illustrates a prosthesis' adapter in perspective, which adapter is to be found in the units in FIGS. 2 and 3 and is schematically illustrated in FIG. 1C,

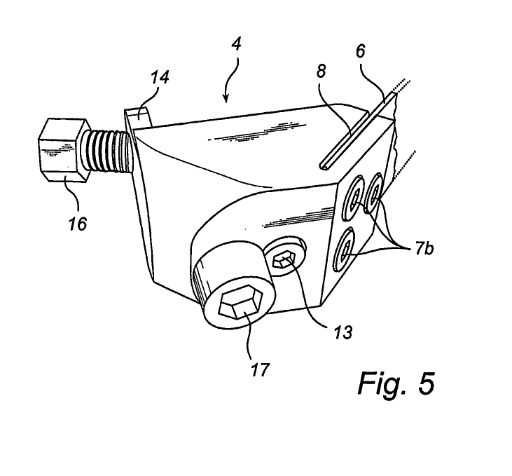

FIG. 5 illustrates the prosthesis adapter in FIG. 4 in perspective from the opposite side,

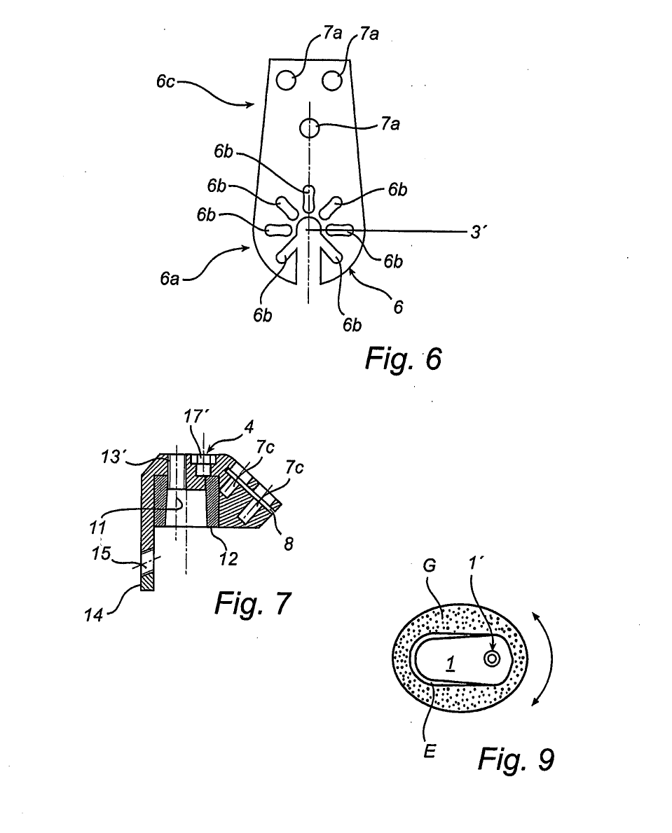

FIG. 6 illustrates a holder for the prosthesis adapter,

FIG. 7 shows the prosthesis adapter in FIGS. 4 and 5 partly in section,

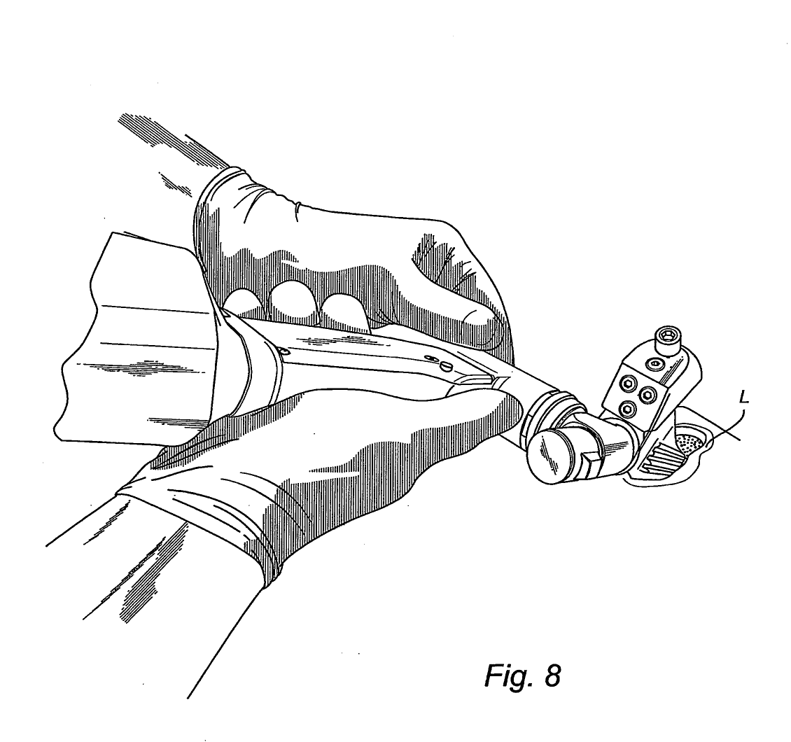

FIG. 8 shows the configuration of the prosthesis insertion unit in FIG. 2 in use for driving a femoral prosthesis into the shaft of a femur, and

FIG. 9 is a cross-sectional view of the upper part of a femoral prosthesis stem inserted into a grain bed, in cross-section.

The prosthesis insertion unit in FIG. 1 comprises a femoral prosthesis 1, a vibrating tool 2 with a vibrating head 3, and an adapter 4 which connects the vibrating tool 2 to the prosthesis 1, via the vibrating head 3.

The vibrating head 3 is arranged to turn, oscillate, back and forth, about an axis 3′ by means of a motor included in the tool 2.

Such vibrating tools are known, of which an example, the vibrating tool 2, is manufactured by Linvatec Corporation, USA, under the name Hall® Power Pro® 5300. This exemplary tool is intended to connect an elongate flat bone saw blade to the head 3, for oscillating the saw blade in the plane of the saw blade back and forth about the axis 3′, rigidly connected to the head 3 and perpendicular to the axis 3′. The teeth of the saw blade are positioned on the other free end, opposite to the connecting end, of the saw blade. The head 3 is divided in two and the two parts 3a and 3b can be separated along the axis 3′ to accommodate the connecting end of the saw blade, and again be put together to lock the saw blade in the head 3. There is an operating knob 5 for said separating and putting together. The vibrating tool 2 which is known per se is, as is evident from several figures, in the form of a pistol with a handle and trigger and is battery operated. The oscillation or swivelling axis 3′ is largely parallel to the handle.

The parts 3a, 3b of the head 3 have pins (not shown) on their sides facing each other for engagement with holes arranged in a circle at the connecting end of the saw blade, thus allowing the saw blade to be connected and locked to the head 3 in the direction of different radii (given by the holes) from the axis 3′ to the holes. This arrangement is evident from FIG. 6, which shows an adapter holder 6 which belongs to the unit according to the invention. The holder 6 is slightly elongate and plate-shaped and adapted to be connected to the head 3 at a connecting end 6a which thus has the same design as the prior-art saw blade for connection to the vibrating tool 2. The fixing holes arranged in a circle at the connecting end 6a are designated 6b.

The holder 6 is arranged to rigidly connect the prosthesis adapter 4 to the head 3, which adapter 4 in turn is arranged to rigidly connect to itself the femoral prosthesis 1, so that the swivelling motion of the head 3 is converted into an oscillating motion of the holder 6 back and forth in its plane about the axis 3′, and into an oscillating swivelling motion back and forth of the prosthesis 1 about the longitudinal axis 1′ of the prosthesis 1.

To this end, the prosthesis adapter 4 is rigidly attachable to the other end portion 6c of the holder 6 (i.e. opposite to the connecting end 6a) by a suitable means, here by a number of bolt joints 7a, 7b, 7c (see especially FIGS. 5 and 6). Preferably, this other end portion 6c is inserted into a slot 8 in the adapter (see especially FIGS. 4, 5 and 7).

The prosthesis adapter 4 is designed for rigid connection of the tool 2 and the femoral prosthesis 1 in such a manner that the swivelling axis 3′ of the head 3 and the longitudinal axis 1′ of the prosthesis substantially coincide. It should be noted that by the expression “femoral prosthesis” is here meant a femoral prosthesis of a conventional kind, that is a femoral prosthesis with a stem 1a tapering conically from proximal to distal, sometimes with a curve on its anterior side, and having a projecting upper neck 1b—terminated with a conical head 1c—which is inclined to the longitudinal axis 1′ of the prosthesis to allow the prosthesis to be anatomically correctly connected to the femoral cup of an artificial hip joint. It should also be noted that by prosthesis longitudinal axis 1′ is meant an axis in the longitudinal direction of the prosthesis 1, substantially through the centre of all cross-sections through the stem of the prosthesis, which overlap each other in the longitudinal direction of the prosthesis (as shown in FIG. 1C and FIG. 9). By “substantially” just mentioned and “substantially coinciding”—relating to the position of the axes 1′ and 3′ relative to each other—in description and claims—are meant on the one hand parallelism between the axes 1′ and 3′, the axis 3′ of the vibrating head 3 which is positioned at a distance a maximum (see FIG. 1C) from the axis 1′, the axis 3′ still extending through the major part of the stem of the prosthesis, or an angle between the axes 1′ and 3′ of 15° maximum, preferably 10° maximum and most advantageously 5°. Moreover the surface of the prosthesis 1 is preferably rough. Advantageously the prosthesis 1 or the prosthesis stem 1a further has an upper means 9 for engaging an instrument to extract the prosthesis from the femur, if the prosthesis should have been implanted incorrectly or has to be extracted from the femur in connection with reoperation. Such a means 9 is available in most of the femoral prostheses available on the market, but can also within the scope of the invention be arranged on the prosthesis, if the prosthesis has no such means.

In the embodiment described, the means 9 is a conventional bottom hole arranged in the upper portion of the prosthesis stem 1a, that is on the prosthesis neck 1b (see FIGS. 1A and 1B).

The adapter 4 has a conical recess 11 (see FIGS. 4 and 7) designed to allow reception, with a tight fit, of the conical neck head 1c (for engagement with a femoral ball) of the femoral prosthesis 1. The recess 11 is here formed in a separate sleeve 12, inserted in a space in the adapter and attached thereto by means of a bolt 13 (the threaded bolt hole is designated 13′ in FIG. 7). The arrangement with a separate sleeve 12 allows replacement of a sleeve 12 with a certain inner dimension (diameter/tapering angle) with a sleeve 12 with another inner dimension for adjustment to prostheses 1 with different conical neck heads 1c.

To secure the prosthesis 1 in the recess 11, the adapter 4 is formed with a lug 14 with a threaded hole 15, through which a bolt 16 with its screw end can engage the prosthesis 1 just below its neck head 1c, inserted in the recess 11, sideways so that when the bolt 16 has been tightened a force component exerts a pressure on the prosthesis towards the recess 11. The prosthesis is engaged by the screw end of the bolt 16 in/on the prosthesis extracting means, that is in this case the extraction hole 9. In this example the lug 14 is, for this purpose, largely parallel to the recess 11 and the threaded hole 15 is inclined towards the recess 11. A screw 17 inserted in the adapter 4 (see FIGS. 3 and 5; the screw hole is designated 17′ in FIG. 7) can be made to engage the conical head 1c of the prosthesis 1 to press the neck head 1c from the recess 11 and, thus, the prosthesis 1 from the adapter 4, if the neck head 1c should fit tightly in the recess 11. Such loosening may be convenient, for instance, when the prosthesis 1 is completely driven into the shaft of the femur.

As mentioned above, it is an important feature of the invention that, when the prosthesis 1, the vibrating tool 2 and the adapter 4 have been connected to each other in the fashion described above, the axis 3′ of the oscillating head 3 is essentially aligned with the longitudinal axis 1′ of the prosthesis 1, see FIGS. 2 and 3.

It will now be obvious that the prosthesis 1, connected to the vibrating tool via the adapter 4, can be driven down into a grain bed in the shaft of the femur in an oscillating spiral motion along a circular arc, the axis 3′ of the spiral motion being essentially aligned or coinciding with the longitudinal axis 1′ of the prosthesis 1, the enclosed angle of the circular arc, that is the amplitude of the striking angle of the prosthesis stem, being determined by the striking angle of the vibrating tool. Said spiral motion is, due to the rigid connection between the vibrating tool and the prosthesis, to be controlled by the surgeon all the time during driving down. The spiral motion, rather than the oscillating motion of the prosthesis according to prior art, minimises the risk of grains being scattered about at the site of surgery (this can be expressed in such a manner that while the grains boil during driving down of the prosthesis according to prior art, FIG. 1A, they simmer during driving down according to the inventive technique). The generation of heat due to friction between different parts of the chain from the vibrating tool to the prosthesis is minimised. Moreover essentially uniform packing of the grains along the length of the prosthesis stem, compared with prior art, is obtained, which results in significantly improved resistance to extraction, that is a significantly smaller risk of the prosthesis coming loose.

The enclosed angle of said circular arc, that is the amplitude of the striking angle of the prosthesis stem, determined by the vibration (oscillation) striking angle of the vibrating tool, should be small, so that no substantial vacant space E occurs in the upper flat part of the prosthesis, see FIG. 9, where G designates the grain bed. The above-described prior-art vibrating tool 2 has a striking angle of 4.5°. Testing of other striking angles has demonstrated that the inventive technique produces better results than prior art (the technique according to FIG. 1A) in terms of no substantial scattering about of grains, and uniform packing of grains along the prosthesis stem is also achieved with striking angles in the range of 2-10°.

In the configuration in FIG. 2, the adapter holder 6 has been oriented essentially along the pistol barrel, while in the configuration in FIG. 3, it has been oriented essentially transversely to the pistol barrel. FIG. 8, which shows the prosthesis insertion unit in use, that is during driving down of a femoral prosthesis into a femur L, shows the same configuration as FIG. 3. As will be obvious from that stated above and from FIG. 6, other configurations are obtained by choosing other engagements between the pins (not shown) of the oscillation head 3 and the holes 6b. For instance a comfortable working position for the operator can determine the choice.

The vibrating tool, as well as the adapter and its holder, need, of course, not have the indicated design; the important thing is that the vibration (oscillation) motion of the vibrating tool about an axis 3′ can be transferred, possibly via a suitable adapter designed for the purpose, to the prosthesis, the axis 3′ essentially coinciding with the longitudinal axis 1′ of the prosthesis. The vibrating tool is preferably of a type having a variable frequency of the oscillation/vibration. The vibrating tool described above has a variable frequency of up to 11500 strokes/min. U.S. Pat. No. 5,015,256 prefers the use of two different vibration frequencies during driving down, as mentioned above: initially a high frequency and close to the end a low frequency, before striking with a hammer on the prosthesis for final fixing thereof in the grain bed. The technique according to the invention does not have the same preference for two different frequencies, but, as mentioned above, has the option of varying the frequency of the vibrating tool, should the surgeon for some reason want to use such a variation during driving down. Also when driving down a prosthesis into a cavity substantially filled with grains using the inventive technique, a final blow of the hammer on the prosthesis is preferred for final fixing thereof in the grain bed.

It will also be obvious that the adapter 6 need not have the shown design, as long as the above requirements substantial linearity and rigid connection are maintained.

A femoral prosthesis has been described above as an example of the application of the invention. It will be appreciated, however, that the invention is applicable to other elongate prostheses for insertion into corresponding elongate body cavities, such as finger prostheses, arm prostheses, dental prostheses. Besides, the invention is applicable to the same grains as described in U.S. Pat. No. 5,015,256, the character of these grains being described by way of introduction.

The fixation of the prosthesis in the bed can be further improved according to a technique which is disclosed in a copending patent application, filed by the same applicant, by the bone canal not being filled all the way with grains but is allowed to have a vacant space, about 1 cm under the trochanter, and the space which is thus free of grains between prosthesis and bone wall is filled with a substantially continuous ring of a flexible, plastic or non-essentially elastic material. This is carried out before the final blow on the prosthesis in its longitudinal direction for final fixing thereof in the bed and the bone cavity. The ring is compressed and compacted in the space by means of, for instance, a chisel (the ring space tapers distally) so as to exactly fill out the ring space and frictionally engage the prosthesis. The final blow/blows is/are not performed until at this stage. The blow/blows causes/cause the ring to be carried along by the prosthesis, whereby the ring acts like a piston on the uppermost (proximal) portion of the grain bed so as to compact this portion. This action, combined with the strong friction between prosthesis and ring, helps to improve the extraction resistance and also, for instance, the resistance to rotation of the prosthesis. The ring is preferably made of a sheet of a thin, flexible, plastic or non-essentially elastic biocompatible material, preferably titanium, which sheet is deformed, for instance wound, twisted, turned or crumpled up to an elongate formation, which is bent as a ring. The ring also prevents breaking up of the upper proximal part of the bed in connection with the blows for final fixing.

The grains are irregular, essentially plastic or essentially non-elastic.

1. A device for fixing an elongate prosthesis part, such as a stem of a femoral prosthesis, to living bone tissue, which defines a cavity in which a length of the prosthesis is received with a gap to the boundary of the cavity, substantially the entire cavity being occupied by loose but packed grains, irregular in shape, of a material which is accepted by bone tissue, which grains are essentially plastic or non-essentially elastic and lock each other, characterised in that the grains are uniformly packed along the length of the prosthesis part in the cavity.

2. A method of inserting an elongate prosthesis into a prosthesis fixing device, which comprises a bed of grains of biocompatible material, a vibrating tool being used for insertion, the vibrating head of which performs an oscillating motion back and forth about a swivelling axis (3′), characterised in that said swivelling axis (3′) of the vibrating tool is allowed to essentially coincide with the longitudinal axis (1′) of the elongate prosthesis (1).

3. A prosthesis insertion unit for inserting an elongate prosthesis into an elongate body cavity substantially in the longitudinal direction thereof, said body cavity being substantially filled with a bed of grains of biocompatible material, characterised in that it comprises
(A) a motor-driven (2) vibrating head (3) arranged to oscillate in a swivelling motion back and forth about a swivelling axis (3′),
(B) the prosthesis (1) and
(C) an adapter (4, 6) designed to rigidly connect the oscillation head (3) and the prosthesis (1) to each other in such a manner that the swivelling axis (3′) of the oscillation head (3) and the longitudinal axis (1′) of the prosthesis (1) essentially coincide with each other.

4. A prosthesis insertion unit as claimed in claim 3, characterised in that said axes (3′, 1′) make an angle to each other, the deviation from the coincidence of said axes (3′, 1′) being 10° maximum.

5. A prosthesis insertion unit as claimed in claim 3, characterised in that said axes (3′, 1′) are parallel to each other, the swivelling axis (3′) of the oscillation head (3) extending through the major part of the prosthesis seen in the longitudinal direction of the prosthesis.

6. A prosthesis insertion unit as claimed in claim 3, characterised in that the oscillation (striking) angle is 15° maximum, more preferably 2-10° and most advantageously 2-5°.

7. A prosthesis insertion unit as claimed in claim 3, characterised in that the adapter (4, 6) is arranged to connect the prosthesis (1) to the oscillation head (3) of the vibrating tool via a hole (9), which is arranged in the proximal portion of the prosthesis and which is engaged by a screw (16) attached to the adapter (4).

8. A prosthesis insertion unit as claimed in claim 3, in the form of a femoral prosthesis with a neck (1b) and a conical head (1c), characterised in that the screw (16) engages in an extraction hole (9) in the prosthesis neck (1b).

9. A prosthesis insertion unit as claimed in claim 8, characterised in that the adapter (4) has a recess (11) for receiving the conical head (1c) of the prosthesis.

10. A prosthesis insertion unit as claimed in claim 8, characterised in that the recess (11) is formed by a sleeve (12) which is releasably arranged on the prosthesis.

11. A prosthesis insertion unit as claimed in claim 3, characterised in that the vibrating tool (1) is a bone saw, and that the adapter (4) is attached to the oscillation head (3) of the bone saw by a holder (6), whose
